(19) United States
(12) Patent Application Publication
(10) Pub. No.: US 2022/0133662 A1
(43) Pub. Date: May 5, 2022

(54) ORAL DRUG, ADJUVANT CANCER THERAPY, AND PET THERAPEUTIC DIET

(71) Applicants: Takumi SATOH, Tokyo (JP); SCHOOL CORPORATION, AZABU VETERINARY MEDICINE EDUCATIONAL INSTITUTION, Kanagawa (JP)

(72) Inventors: Takumi SATOH, Tokyo (JP); Tadashi YAMASHITA, Kanagawa (JP); Masaki NAGANE, Kanagawa (JP)

(21) Appl. No.: 17/542,984

(22) Filed: Dec. 6, 2021

Related U.S. Application Data
(63) Continuation of application No. PCT/JP2020/023042, filed on Jun. 11, 2020.

(30) Foreign Application Priority Data
Jun. 13, 2019 (JP) 2019-129635
Oct. 24, 2019 (JP) 2019-205075

(51) Int. Cl. A61K 31/19; A61K 9/00; A61P 35/00; A61P 1/14; A23K 20/105; A23K 50/40; A23L 33/10; A23L 33/00

(52) U.S. Cl. CPC A61K 31/19; A61K 9/0053; A61P 35/00; A61P 1/14; A23V 2002/00; A23K 50/40; A23L 33/10; A23L 33/40; A23K 20/105

(57) ABSTRACT

An oral drug includes powdered poly (R)-3-β-hydroxybutyric acid having a weight-average molecular weight of 10,000 to 700,000. The oral drug includes the powdered poly (R)-3-β-hydroxybutyric acid having (a) a purity of 70% or more and (b) a weight-average molecular weight of 10,000 to 590,000. The oral drug includes the powdered poly (R)-3-β-hydroxybutyric acid having (a) a purity of 90% or more and (b) a weight-average molecular weight of 10,000 to 590,000. An oral drug for increasing a ketone body concentration in blood that includes powdered poly (R)-3-β-hydroxybutyric acid having a weight-average molecular weight of 10,000 to 700,000. According to the present disclosure, an increased ketone body concentration in the blood is sustained for a longer period of time.

### CROSS-REFERENCE TO RELATED APPLICATIONS

The present application is a continuation application of International Application number PCT/JP2020/23042, filed on Jun. 11, 2020, which claims priority under 35 U.S.C § 119(a) to Japanese Patent Application No. 2019-129635, filed on Jun. 13, 2019 and Japanese Patent Application No. 2019-205075, filed on Oct. 24, 2019. The contents of these applications are incorporated herein by reference in their entirety.

### BACKGROUND OF THE INVENTION

An oral drug, an adjuvant cancer therapy, and a pet therapeutic diet using ketone donors are provided.

In recent years, various physiological effects of ketone donors such as ketone bodies and ketone ester have attracted attention. Ketone donors have been reported to be effective in promoting decomposition of fat, reducing insulin resistance in diabetes, ameliorating cognitive dysfunctions due to Alzheimer's disease and Parkinson's disease, and the like (Ketogenic dietary therapies for epilepsy and beyond. deCampo D M, Kossoff E H. Curr Opin Clin Nutr Metab Care. 2019 July., and Rho J M, Shao L R, Stafstrom C E. 2-Deoxyglucose and Beta-Hydroxybutyrate: Metabolic Agents for Seizure Control. Front Cell Neurosci. 2019 Apr. 30; 13:172.).

Ketone bodies are hydrolyzed by hydrolases in the small intestine of a mammal and quickly absorbed from the small intestine epithelium to increase a ketone body concentration in the blood. There is a problem in that ketone bodies cannot sustain an increased ketone body concentration in the blood for a long time (Japanese Unexamined Patent Application Publication (Translation of PCT Application) No. 2015-514104, Stubbs B J, Cox P J, Evans R D, Santer P, Miller J J, Faull O K, Magor-Elliott S, Hiyama S, Stirling M, Clarke K. On the Metabolism of Exogenous Ketones in Humans. Front Physiol. 2017 Oct. 30; 8:848., and Stubbs B J, Cox P J, Evans R D, Cyranka M, Clarke K, de Wet H. A Ketone Ester Drink Lowers Human Ghrelin and Appetite. Obesity (Silver Spring). 2018 February; 26(2):269-273).

### BRIEF SUMMARY OF THE INVENTION

The present disclosure focuses on these points, and an object of the present disclosure is to provide an oral drug, an adjuvant cancer therapy, and a pet therapeutic diet using ketone donors capable of sustaining an increased ketone body concentration in the blood for a longer period of time.

A first aspect of the present disclosure provides an oral drug including powdered poly (R)-3-β-hydroxybutyric acid having a weight-average molecular weight of 10,000 to 700,000.

A second aspect of the present disclosure provides an oral drug for increasing a ketone body concentration in blood including powdered poly (R)-3-β-hydroxybutyric acid having a weight-average molecular weight of 10,000 to 700,000.

A third aspect of the present disclosure provides an oral drug for activating intestinal bacteria in the large intestine including powdered poly (R)-3-β-hydroxybutyric acid having a weight-average molecular weight of 10,000 to 700,000.

A fourth aspect of the present disclosure provides an oral drug for activating macrophages in the large intestine including powdered poly (R)-3-β-hydroxybutyric acid having a weight-average molecular weight of 10,000 to 700,000.

A fifth aspect of the present disclosure provides an adjuvant cancer therapy including powdered poly (R)-3-β-hydroxybutyric acid having a weight-average molecular weight of 10,000 to 700,000.

A sixth aspect of the present disclosure provides a pet therapeutic diet, to be provided to subjects during or after treatment of cancer, including powdered poly (R)-3-β-hydroxybutyric acid, having a weight-average molecular weight of 10,000 to 700,000.

### BRIEF DESCRIPTION OF THE DRAWINGS

FIG. 1 shows a mechanism of oral administration of PHB.

FIG. 2 shows an overview of PHB hydrolysis by enzymes of intestinal bacteria.

FIG. 3 shows a mechanism of absorbing PHB.

FIG. 4 shows an overview of a pathway of PHB activating the intestinal bacteria.

FIG. 5 schematically shows a bacterium accumulating PHB granules.

FIG. 6 shows an outline of a plurality of methods for purifying PHB.

FIG. 7 is a flowchart showing an outline of the method for purifying powdered PHB of the present disclosure.

FIG. 8 shows measurement results of a ketone body concentration in the blood.

FIG. 9 shows measurement results of the ketone body concentration in the blood after ingesting plain yogurt containing powdered PHB.

FIG. 10 shows measurement results of the ketone body concentration in the blood when yogurt containing powdered PHB was ingested daily.

FIGS. 11A to 11D show survival rates of COS7 cells when ketone bodies (HB) or ketone ester (KE) was added.

FIGS. 12A to 12D show survival rates of Hela cells when ketone bodies (HB) or ketone ester (KE) was added.

FIG. 13 shows measurement results of the ketone body concentration in the blood in mice fed with powdered PHB.

FIGS. 14A and 14B show a confirmation test of an inhibitory effect of PHB on cancer growth.

FIGS. 15A and 15B are graphs showing the inhibitory effect of PHB on cancer growth.

### DETAILED DESCRIPTION OF THE INVENTION

Hereinafter, the present invention will be described through exemplary embodiments of the present invention, but the following exemplary embodiments do not limit the invention according to the claims, and not all of the combinations of features described in the exemplary embodiments are necessarily essential to the solution means of the invention.

As the world becomes increasingly concerned about global environmental issues, there is a growing interest in biodegradable plastics that can be completely degraded in nature. The present inventors focused on the fact that some microorganisms accumulate granules of poly(R)-3-hydroxybutyric acid (hereinafter referred to as PHB) used as biodegradable plastics, and investigated the use of powdered PHB for the purpose of treating diseases such as cancer, allergic disease, autoimmune disease, inflammatory bowel disease, constipation, and the like.

The present disclosure provides an oral drug containing PHB having a weight-average molecular weight of 10,000 to 700,000. Ketone bodies (referred to as (R)-3-hydroxybutyric acid or HB) and ketone ester (referred to as KE) is known as ketone donors that release ketone in the gastrointestinal tract of a mammal, in addition to PHB. Hereinafter, PHB refers to poly(R)-3-β-hydroxybutyric acid, and ketone bodies refer to (R)-3-β-hydroxybutyric acid. Ketone bodies and ketone ester is classified as type I ketone donors. On the other hand, PHB is classified as a type II ketone donor. When PHB is orally ingested, a ketone body concentration in the blood increases at least about 6 hours after ingestion. PHB has an advantage that the effect of increasing the ketone body concentration in the blood is sustained longer than in the case of ketone bodies or ketone ester.

Poly(R)-3-β-hydroxybutyric acid (hereinafter also referred to as PHB) is represented by the following chemical formula (1).

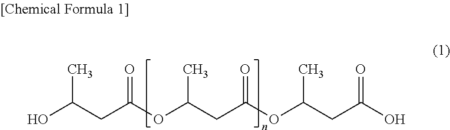

### [Mechanism of Oral Administration of PHB]

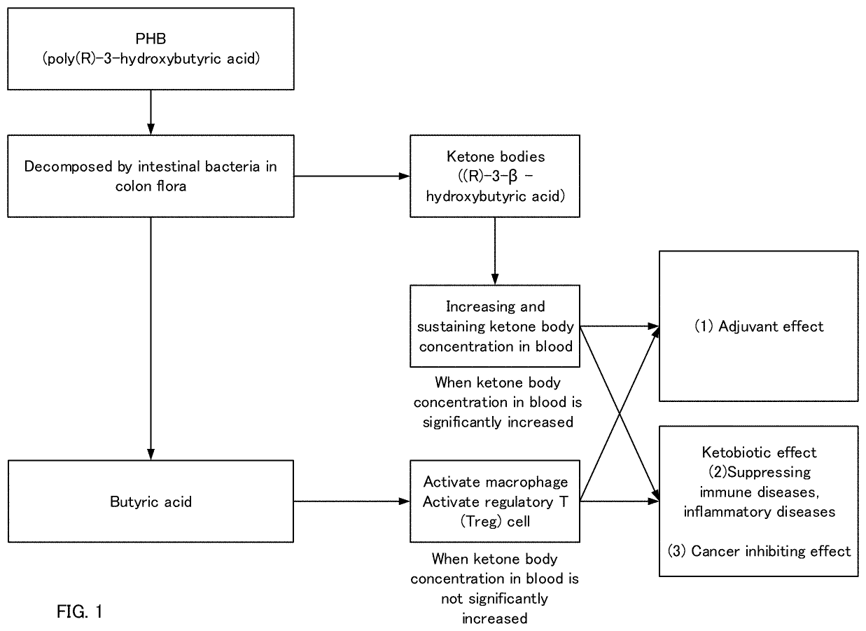

FIG. 1 shows a mechanism of oral administration of PHB. PHB (poly(R)-3-hydroxybutyric acid) is decomposed by the intestinal bacteria in the colon flora (bacteria belonging to Clostridium cluster IV, Clostridium cluster XIVa, and Clostridium cluster XVIII). As a result, the intestinal bacteria produce ketone bodies ((R)-3-hydroxybutyric acid) or butyric acid. The produced ketone bodies are absorbed from the large intestine epithelium such that the ketone body concentration in the blood increases significantly. Further, when PHB is repeatedly administered orally, an increased ketone body concentration in the blood is sustained.

When the ketone body concentration in the blood is significantly increased, another method for treating cancer can be applied to obtain the adjuvant effect that inhibits the growth of cancer cells ((1) in FIG. 1). When the ketone body concentration in the blood is significantly increased, the intestinal bacteria decompose PHB to produce ketone bodies, thereby increasing the number of the intestinal bacteria and obtaining the ketobiotic effect in which the amount of ketone bodies released into the intestine is increased. Examples of the ketobiotic effect include (a) an effect of suppressing immune diseases, inflammatory diseases, and the like ((2) in FIG. 1) and (b) a cancer inhibiting effect in which ketone bodies alone inhibit the growth of cancer cells ((3) in FIG. 1).

On the other hand, when a relatively small amount of PHB is administered orally, the ketone body concentration in the blood does not increase significantly. At this time, the butyric acid produced by the intestinal bacteria in the colon flora activates macrophages that reside in Peyer's patches in the intestine. The activated macrophages activate naive T cells to be differentiated into regulatory T (Treg) cells. Regulatory T cells are involved in the adjuvant effect that inhibits the growth of cancer cells by applying another cancer treatment method. Regulatory T cells suppress autoimmune diseases, inflammatory diseases, and the like by modulating the immune mechanism in the body. Further, regulatory T cells are also involved in cancer inhibitory action that inhibits the growth of cancer cells by ketone bodies alone. Therefore, even if the ketone body concentration in the blood is not significantly increased by the oral administration of PHB, the similar adjuvant effect and the ketobiotic effect can be obtained in the same manner as when the ketone body concentration in blood is significantly increased.

### [Mechanism of Absorbing PHB]

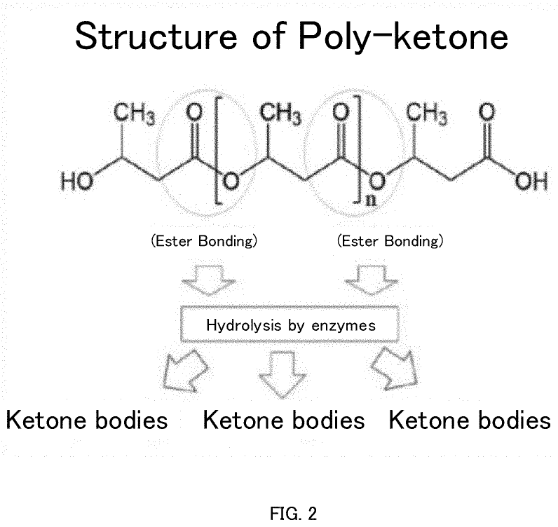

PHB is a compound (average polymerization degree of about 2,000) that is a resultant of ketone bodies becoming a polymer due to ester bonding. PHB has extremely low hydrophilicity. FIG. 2 shows an overview of PHB hydrolysis by enzymes of intestinal bacteria. Since the ester bonding of PHB cannot be hydrolyzed by the mammalian esterase, PHB is not absorbed in the small intestine. The ester bonding of PHB is hydrolyzed by the enzymes of the intestinal bacteria in the large intestine to produce ketone bodies.

Examples of the intestinal bacteria including the enzymes capable of hydrolyzing PHB include bacteria belonging to o_Clostridiales or o_Erysipelotrichales of p_Firmicutes, and o_Bacteroidales of p_Bacteroidetes. Examples of the enzymes capable of hydrolyzing PHB include alkaline lipases derived from the genus Cromobacter, lipoprotein lipases derived from the genus Alkaligenus, lipases derived from the genus Pseudomonas, lipases derived from the genus Candida, lipases derived from the genus Mocol, lipases derived from the genus Rhizopus, lipases derived from the genus Penicillium, lipases derived from the genus Phycomyces, and the like (Japanese Unexamined Patent Application Publication No. 2010-168595). The enzymes capable of hydrolyzing PHB preferably include bacterial groups belonging to Clostridium cluster IV, Clostridium cluster XIVa, and Clostridium cluster XVIII, which are capable of producing butyric acid.

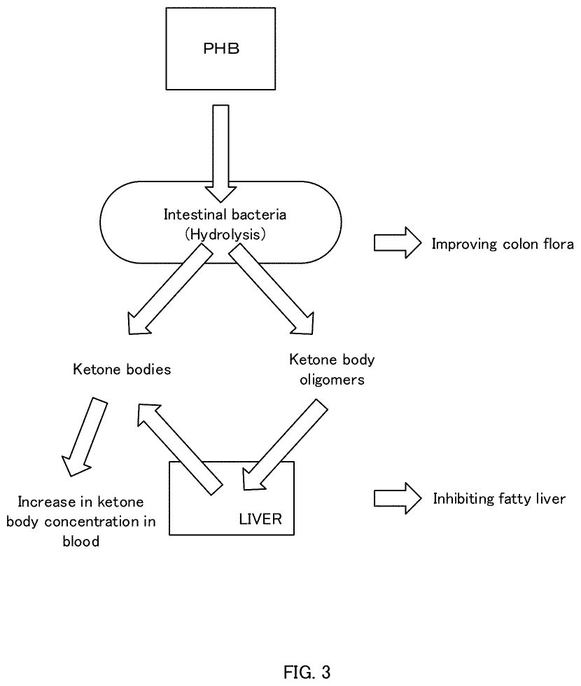

FIG. 3 shows a mechanism of absorbing PHB. PHB provides physiological effects in a plurality of pathways. In an initial pathway, PHB is decomposed into ketone bodies by the lipases of the intestinal bacteria in the large intestine and absorbed from the large intestinal epithelium to contribute to an increase in the ketone body concentration in the blood. Since the process in which the powdered PHB is decomposed by the intestinal bacteria of the large intestine to produce ketone bodies requires a long time, it is considered that the time during which the ketone body concentration in the blood is high is sustained longer than that in the case where ketone bodies or ketone ester is administered orally.

When PHB is hydrolyzed by the intestinal bacteria, not only ketone bodies but also ketone body oligomers consisting of 3 to 10 molecules of ketone bodies are produced. Ketone body oligomers are absorbed from the large intestinal epithelium, brought to the liver, and hydrolyzed into ketone bodies in the liver (Japanese Unexamined Patent Application Publication No. 2010-168595, PCT International Publication No. 2005/021013, and PCT International Publication No. 2019/035486). Said ketone oligomers become direct sources of nutrition in mitochondria of liver cells, and are therefore assumed to have a strong inhibitory effect on fatty liver, which is the most fundamental cause of lifestyle-related diseases.

When PHB is hydrolyzed by the intestinal bacteria, PHB is considered to become a nutrient substrate for the intestinal bacteria first. The oral drugs containing PHB are administered for the purpose of activating the intestinal bacteria in the large intestine. PHB has the potential to induce a variety of physiological effects, starting with improvement of the colon flora. PHB causes the production of ketone bodies in the intestinal bacteria and activates the intestinal bacteria via ketone bodies. At this time, the ketone bodies produced by the intestinal bacteria are absorbed from the large intestinal epithelium to increase the ketone body concentration in the blood. The inventors call this “ketobiotic.”

If the weight-average molecular weight of PHB is less than 10,000, the ketone body concentration in the blood cannot be continuously increased. On the other hand, if the weight-average molecular weight of PHB exceeds 700,000, it is considered that it takes too much time to increase the ketone body concentration in the blood since PHB requires time to be hydrolyzed by the intestinal bacteria. For this reason, the weight-average molecular weight of PHB is preferably between 10,000 and 700,000, for example.

PHB is synthesized using bacteria, for example. It is possible to chemically synthesize PHB by increasing the polymerization degree of ketone bodies using an asymmetric catalyst, but this cannot synthesize PHB having a weight-average molecular weight exceeding 10,000. Therefore, bacteria must be used to synthesize PHB having a weight-average molecular weight exceeding 10,000.

The weight-average molecular weight of PHB varies depending on the bacteria used for synthesis. For example, PHB derived from the genus Listeria has a weight-average molecular weight exceeding 700,000 (purity: about 26%), whereas PHB derived from the genus Halomonas has a weight-average molecular weight of 590,000 or less (purity: 70% or less). It is believed that the higher the weight-average molecular weight of PHB, the longer the time required for hydrolyzing in the intestinal bacteria. The weight-average molecular weight of PHB to be administered orally to dogs and humans is more preferably in the range of 10,000 to 590,000 such that the ketone body concentration in the blood increases within several hours after administration. Therefore, it is practically preferable to use PHB derived from the genus Halomonas. It is considered that high-pressure sterilization during the extraction of PHB partially decomposes PHB and lowers its weight-average molecular weight (PCT International Publication No. 2019/035486).

The purity and weight-average molecular weight of the powdered PHB are considered to influence the time required to increase the ketone body concentration in the blood (PCT International Publication No. 2008/120778). The purity of the powdered PHB is 50% or more, for example. The powdered PHB with a purity of 50% or more is relatively easy to mass-produce, and can provide a sustained increase in the ketone body concentration in the blood. In order to enhance the effect of the powdered PHB on increasing the ketone body concentration in the blood, the purity of the powdered PHB may be set to 70% or higher. In order to further enhance the effect of the powdered PHB on increasing the ketone body concentration in the blood, the purity of the powdered PHB may be set to 90% or higher.

For example, the purity of PHB can be increased up to 100% by Soxhlet extraction using chloroform. However, Soxhlet extraction cannot be used for commercializing PHB as food or pet food because of the risk of chloroform residue in the final product. Therefore, a purification method combining autoclaving and adding of surfactants is considered to purify PHB having a purity of 70% or more (PCT International Publication No. 2019/035486).

### [Properties of Ketone Bodies]

A ketone body has a structure represented by the following formula (2).

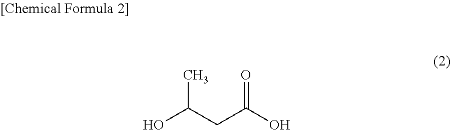

Ketone bodies are produced by hydrolyzing PHB by the enzymes of the intestinal bacteria. Ketone bodies are highly hydrophilic. Since ketone bodies are weak acids, about the same as acetic acid, sodium or arginine salts are usually used. Ketone salts can be orally ingested. However, care should be taken not to intake too much sodium when administering oral drugs containing sodium salts of ketone bodies.

In order to produce the ketone salts, it is necessary to precipitate the ketone salts from an aqueous solution, but the cost of precipitating the ketone salts from the aqueous solution is high due to the very high hydrophilicity of ketone bodies. The ketone salts are easily ionized in the aqueous solution. Ketones bodies exist mostly as ions in the environment of the small intestine, which is weakly alkaline, and are rapidly absorbed into the body by a specific monocarboxylic acid transporter. When ketone bodies are ingested, the ketone body concentration in the blood increases within a few minutes (Japanese Unexamined Patent Application Publication No. 2018-166481, and Japanese Unexamined Patent Application Publication No. 2018-000073). Examples of the physiological functions of ketone bodies include the following (Potential Synergies of 3-Hydroxybutyrate and Butyrate on the Modulation of Metabolism, Inflammation, Cognition, and General Health. Cavaleri F, Bashar E. J Nutr Metab. 2018 Apr. 1, and β-Hydroxybutyrate: A Signaling Metabolite. Newman J C, Verdin E. Annu Rev Nutr. 2017 Aug. 21; 37:51-76.):

(1) Lowering the blood glucose level
(2) Lowering of fatty acids in the blood
(3) Anti-cancer effect
(4) Restraining epileptic seizures
(5) Inhibition of oxidative stress
(6) Inhibition of inflammatory reaction

### [Properties of Ketone Ester]

The formal name of ketone ester is 3-hydroxybutyl-3-hydroxybutyric acid. Ketone ester has a structure represented by the following formula (3).

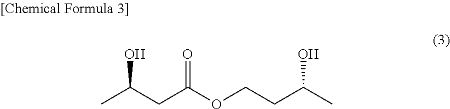

Ketone ester is an ester-bonded form of a ketone body and 1,3-butanediol. Since the ester bonds of ketone ester are rapidly hydrolyzed by the esterase in the small intestine, anions of ketone bodies are produced in the small intestine. Ketone ester is absorbed into the body by the specific monocarboxylic acid transporter, like ketone bodies. Alcohol that is ester-bonded to ketone bodies in ketone ester is oxidized and converted to ketone bodies (PCT International Publication No. 2005/021013, Japanese Unexamined Patent Application Publication No. 2017-071644, and Japanese Unexamined Patent Application Publication No. 2018-138549). Ketone ester, like ketone salts, can act in a short time and increase the ketone body concentration in the blood to several mM in a few minutes. However, the flavor of ketone ester is very poor. Ketone ester is expensive because it must be synthesized by asymmetric synthesis.

### [Comparison of Ketone Donors]

Ketone bodies and ketone ester, among ketone donors, can rapidly increase the ketone body concentration in the blood, whereas PHB requires about five hours to increase the ketone body concentration in the blood. On the other hand, PHB is tasteless and odorless, and thus is easy to be used in pet foods and the like. PHB can be mass-produced using bacteria, which is very advantageous compared to ketone bodies and ketone ester in terms of cost (Japanese Unexamined Patent Application Publication No. 2010-168595, and PCT International Publication No. 2019/035486). PHB is suitable for treatment of chronic diseases such as lifestyle-related diseases because it can continuously increase the ketone body concentration in the blood.

### [Inhibiting Effect of Ketone Bodies on Cancer Growth]

Ketone bodies inhibit the growth of cancer cells and retrogress the cancer. Cancer cells rarely use the mitochondria to obtain energy substrates, but use the glycolytic system to obtain the necessary energy substrates (Warburg effect). A pathway that the mitochondria use to obtain the energy substrates uses organic acids such as ketone bodies, and it is known that when this pathway is dominant and the glycolytic system is restricted, cancer cells either go into apoptosis or stop proliferating.

It has been suggested that ketone bodies may inhibit the proliferation of cancer cells via activation of the receptor HCAR2 in addition to their action as the energy substrates, and it has been reported that ketone bodies inhibit the proliferation of many types of cancer cells (Tumor Metabolism, the Ketogenic Diet and βHydroxybutyrate: Novel Approaches to Adjuvant Brain Tumor Therapy. Woolf E C, Syed N, Scheck A C. Front Mol Neurosci. 2016 Nov. 16; 9:122, and The influence of ketogenic therapy on the 5 R's of radiobiology. Klement R J. Int J Radiat Biol. 2019 April; 95(4):394-407.). Since PHB inhibits cancer growth by continuously increasing the ketone body concentration in the blood, PHB can be considered a ketogenic cancer-inhibitor.

### [Adjuvant Effect of Ketone Bodies]

The oral drugs containing the powdered PHB are administered as adjuvant cancer therapies to be used with another method for treating cancer. Examples of cancer include solid cancer such as gliomas, breast cancer, liver cancer, kidney cancer, gastrointestinal cancer, uterine cancer, prostate cancer, and lung cancer. Of particular interest in the cancer inhibitory action of ketone bodies is the case of the treatment of gliomas. Gliomas have the following two features. One is that they can be treated with radiotherapy due to its low invasiveness. The other is that most anticancer agents have little effect on gliomas because they cannot cross the blood-brain barrier. Ketone bodies have high transferability to the brain, and 20-30% of ketone bodies are transferred to the brain. In this sense, a combination of the radiotherapy and ketone bodies is expected to have a treating effect in clinical field.

For example, irradiation of gliomas in mice significantly improved the survival rates of the mice, and the irradiation plus ketone diets resulted in even greater improvements in the survival rates of the mice. The inhibitory effect of ketone bodies on the growth of cancer cells is observed not only in gliomas, but in many types of cancer cells, such that many countries focus on this adjuvant effect and conduct clinical trials for cancer treatment (The ketogenic diet is an effective adjuvant to radiation therapy for the treatment of malignant glioma. Abdelwahab M G, Fenton K E, Preul M C, Rho J M, Lynch A, Stafford P, Scheck A C. PLoS One. 2012; 7(5):e36197, and Tumor Metabolism, the Ketogenic Diet and β-Hydroxybutyrate: Novel Approaches to AdjuvantBrain Tumor Therapy. Woolf E C, Syed N, Scheck A C. Front Mol Neurosci. 2016 Nov. 16; 9:122.). PHB, when used in combination with other anticancer agents, provides the adjuvant effect that enhances the inhibitory effect of the anticancer agents on cancer growth, as described below.

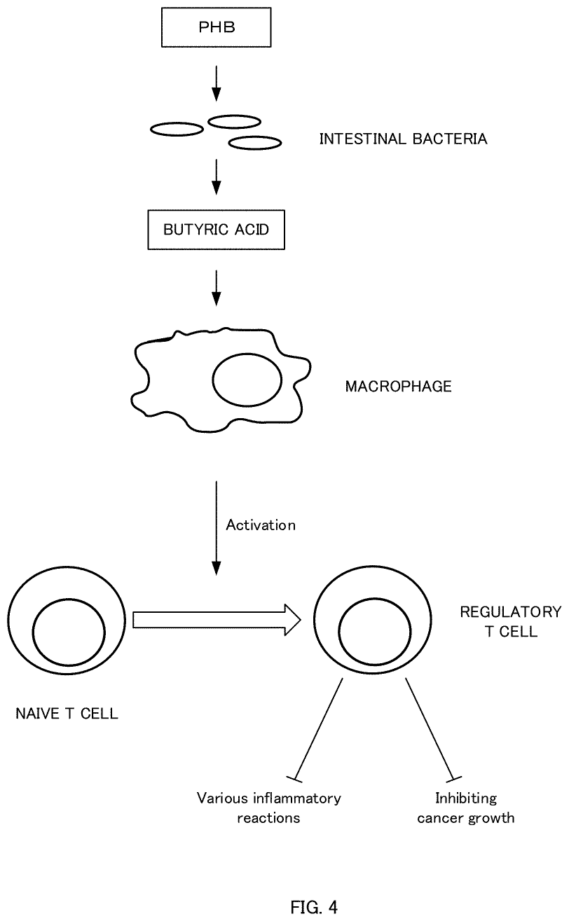

The oral drugs containing the powdered PHB are administered for the purpose of activating macrophages in the large intestine. FIG. 4 shows an overview of the pathway of PHB activating the intestinal bacteria (Singh N, Gurav A, Sivaprakasam S, et al. Activation of Gpr109a, receptor for niacin and the commensal metabolite butyrate, suppresses colonic inflammation and carcinogenesis. Immunity. 2014; 40(1):128-139.). When PHB is incorporated into the intestinal bacteria, the intestinal bacteria activate and metabolically produce the butyric acid. As shown in FIG. 4, the butyric acid activates macrophages residing in the “Peyer's patches” in the large intestine. The activated macrophages activate naive T cells to be differentiated into regulatory T cells. Regulatory T cells inhibit the growth of cancer cells. Further, regulatory T cells inhibit various inflammatory reactions such as allergic diseases, autoimmune diseases, and inflammatory bowel diseases (Japanese Unexamined Patent Application Publication (Translation of PCT Application) No. 2018-532758). Therefore, the oral drugs containing the powdered PHB may provide inhibition of the symptoms of cancer, allergic diseases, autoimmune diseases, and inflammatory bowel diseases.

### [Pet Therapeutic Diet]

Pet therapeutic diets are prescribed by a veterinarian at a veterinary clinic, for example. The pet therapeutic diets contain special foodstuff molecules that help pets recuperate their own strength and other factors during or after treatment of a disease. The pet therapeutic diets include agents and supplements that can be administered orally to help ameliorate the side effects caused by the treatment of pets' diseases. The pet therapeutic diets containing the powdered PHB are provided to a subject to be treated, for example, during or after the treatment of cancer. Further, the pet therapeutic diets containing the powdered PHB may also be provided to a subject to be treated for the treatment of allergic diseases, autoimmune diseases, inflammatory bowel diseases, or constipation.

### [PHB-Applicable Diseases]

The oral drugs containing the powdered PHB are administered for the purpose of increasing the ketone body concentration in the blood. The powdered PHB induces a sustained increase in the ketone body concentration in the blood to provide various physiological effects due to ketone bodies (Ketone Bodies and Exercise Performance: The Next Magic Bullet or Merely Hype? Pinckaers P J, Churchward-Venne T A, Bailey D, van Loon L J. Sports Med. 2017 March; 47(3):383-391, and Ketone body metabolism and cardiovascular disease. Cotter D G, Schugar R C, Crawford P A. Am J Physiol Heart Circ Physiol. 2013 Apr. 15; 304(8):H1060-76.). It has been suggested, from about twenty years ago, that ketone bodies may have an ameliorating effect on a variety of morbid states originating from inflammation, autoimmune allergy, cell death, active oxygen, lipid peroxidation, hyperexcitability of cell membranes, cancer, accumulation of fat, infection, accumulation of abnormal proteins, calcification, circulation disorder, and the like (Ketoacids? Good medicine? Cahill G F Jr, Veech R L. Trans Am Clin Climatol Assoc. 2003; 114:149-6, and Ketone bodies, potential therapeutic uses. Veech R L, Chance B, Kashiwaya Y, Lardy H A, Cahill G F Jr. IUBMB Life. 2001 April; 51(4):241-7.). The oral drugs containing the powdered PHB can be applied to various applicable diseases by increasing the ketone body concentration in the blood. Examples of the applicable diseases to which the oral drugs containing the powdered PHB can be applied include the following: multiple sclerosis, amyotrophic lateral sclerosis, Alzheimer's disease, Parkinson's disease, Huntington's disease, acute haemorrhagic leucoencephalomyelitis, Hurst's disease, encephalomyelitis, optic neuritis, spinal cord lesion, acute necrotizing myelitis, transverse myelitis, chronic progressive myelopathy, progressive multifocal leukoencephalopathy, radiation myelopathy, HTLV-1 associated myelopathy, monophasic isolated demyelination, central pontine myelinolysis, leucodystrophy, inflammatory demyeltnising polyneuropathy, acute Guillain-Barre syndrome, polyneuritis, myasthenia gravis, Eaton Lambert Syndrome, encephalomyelitis, inflammatory bowel disease, Crohn's disease, lupus, systemic Lupus erythematodes, asthma, Leber's disease, Devic's disease, Friedrich's Ataxia, a mitochondrial Central Nervous System disease, scleroderma, uveitis, anti-phospho lipid antibody syndrome, polyarthritis, polyarticular juvenile idiopathic arthritis, sickle cell disease, ankylosing spondylitis, myositis, atherosclerosis, diabetic peripheral neuropathy, head injury, stroke, HIV-dementia, myocardial infarction, angina pectoris, cardiac insufficiency, psoriasis, psoriatic arthritis, Sjogren's syndrome, diabetes, blistering skin diseases, sarcoidosis, osteoarthritis, ulcerative colitis, vasculitis, lung fibrosis, idiopathic pulmonary fibrosis, liver fibrosis, graft-versus-host reactions, Hashimoto's thyroiditis, Grave's disease, pernicious anaemia, hepatitis, neurodermatitis, retinopathia pigmentosa, a mitochondrial encephalomyopathy, osteochondritis syphilitica (Wegener's disease), cutis marmorata (livedo reticularis), Behcet disease, panarteriitis, osteoarthritis, gout, artenosclerosis, Reiter's disease, pulmonary granulomatosis, encephalitis, endotoxic shock (septic-toxic shock), sepsis, pneumonia, anorexia nervosa, Rennert T-lymphomatosis, mesangial nephritis, post-angioplastic restenosis, reperfusion syndrome, cytomegaloviral retinopathy, adenoviral diseases, AIDS, post-herpetic neuralgia, post-zoster neuralgia, mononeuropathia multiplex, mucoviscidosis, Bechterew's disease, Barett oesophagus, Epstein-Barr virus infection, cardiac remodeling, interstitial cystitis, human tumour radiosensitisation, multi-resistance of malignant cells to chemotherapeutic agents, granuloma annulare, a cancer, chronic obstructive pulmonary disease, PDGF induced thymidine uptake of bronchial smooth muscle cells, bronchial smooth muscle cell proliferation, adrenal leukodystrophy, alcoholism, Alper's disease, ataxia telangiectasia, batten disease, bovine spongiform encephalopathy, cerebral palsy, Cockayne syndrome, corticobasal degeneration, Creutzfeldt-Jakob disease, familial fatal insomnia, frontotemporal lobar degeneration, Kennedy's disease, Lewy body dementia, neuroborreliosis, Machado-Joseph disease (Spinocerebellar ataxia type 3), multiple system atrophy, narcolepsy, Niemann Pick disease, Pick's disease, primary lateral sclerosis, Prion diseases, progressive supranuclear palsy, Refsum's disease, Sandhoff disease, Schilder's disease, subacute combined degeneration of spinal cord secondary to pernicious anaemia, spinocerebellar ataxia, spinal muscular atrophy, Steele-Richardson-Olszewski disease, tabes dorsalis, toxic encephalopathy, MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's syndrome, MNGIE (Myopathy and external ophthalmoplegia; Neuropathy; Gastro-Intestinal; Encephalopathy), Kearns-Sayre syndrome, NARP, hereditary spastic paraparesis, mitochondrial myopathy, optic neuritis, progressive multifocal leukoencephalopathy, pyoderma gangrenosum, erosive pustular dermatosis of the scalp, Sweet's syndrome, bowel-associated dermatosis-arthritis syndrome, pustular psoriasis, acute generalized exanthematous pustulosis, keratoderma blenorrhagicum, Sneddon-Wilkinson disease, amicrobial pustulosis of the folds, infantile acropustulosis, transient neonatal pustulosis, neutrophilic eccrine hidradenitis, rheumatoid neutrophilic dermatitis, neutrophilic urticaria, Still's disease, erythema marginatum, unclassified periodic fever syndromes/autoinflammatory syndromes, bullous systemic lupus erythematosus, neutrophilic dermatosis of the dorsal hands (pustular vasculitis), anaphylaxis, allergic disease, allergic rhinitis, allergic asthma, lung cancer, severe asphyxic episodes of asthma, acute lung injury, acute respiratory distress syndrome, ischemia reperfusion injury, septicemia with multiorgan failure, inderteminate colitis, sickle cell crisis, acute chest syndrome, scleroderma lung disease, chronic asthma, radiation-induced fibrosis sarcoidosis, pulmonary hypertension, bronchopulmonary dysplasia (BPD), lung transplant rejection, pulmonary GVHD Complications, Interstitial pneumonia Syndrome (IPS) in transplant recipients, COPD, silicosis, asbestosis, Primary Sclerosing Cholangitis (PSC), alcohol-induced hepatic fibrosis, autoimmune disease, autoimmune hepatitis, Chronic viral hepatitis (HepB,C), Primary biliary cirrhosis (PBC), Non-alcohol Steatohepatitis (NASH), liver transplant rejection, hepatic complications of GVHD, veno-occlusive disease in transplant recipients, Focal Segmental Glomerular Sclerosis (FSGS), IgA nephropathy, renal complications of GVHD (AKI delayed graft function), acute renal failure post CABG (AKI post CABG), Lupus nephritis, hypertension-induced renal fibrosis, HIV-associated nephropathy, peritoneal dialysis-induced peritoneal fibrosis, retroperitoneal fibrosis, idiopathic glomerulosclerosis, kidney transplant rejection, Alport syndrome, restenosis, Subarachnoid hemorrhage (SAH), heart transplant rejection, cosmetic surgery, chronic wounds, burns, surgical adhesions, keloids, donor graft re-epithelialization, myelofibrosis, corneal transplant, LASIX, trabeculectomy, systemic sclerosis, constipation, radiation induced fibrosis, peripatellar fibrosis, dupuytren's contractures, Hodgkin lymphoma, non-Hodgkin lymphoma, lymphosarcoma, lymphoblastoid leukemia, acute lymphatic leukemia, acute myeloic leukemia, chronic myeloic leukemia, chronic lymphatic leukemia, hemangioma, hemangioendothelioma, hemangiopericytoma, hemangiosarcoma, Kaposi sarcoma, osteosarcoma, fibrosarcoma, oesophageal squamous cell carcinoma, pancreatic carcinoma, gastrointestinal tumors, colon carcinoma, rectum carcinoma, stomach carcinoma, lymphangiosarcoma, brain tumors, neuroblastoma, schwannoma, pheochromocytoma, lung carcinoma, head and neck squamous cell carcinoma, melanoma, non-melanoma skin carcinoma, leiomyomas, leiomyosarcomas, mammary carcinoma, ovarian cancer, endometrial carcinoma, bladder carcinoma, cervix carcinoma, renal carcinoma, and prostate carcinoma.

A subject to be treated that is caused to orally ingest a composition containing PHB is not particularly limited as long as it is a living thing that may suffer from the above-mentioned applicable diseases, and is a human or a mammal other than a human, such as a mouse, a rat, a hamster, a guinea pig, a rabbit, a cat, a dog, a horse, a cow, or a pig.

### [Form of Drugs]

The oral drugs according to the present embodiment can be produced by mixing the active ingredient, which is the powdered PHB, with physiologically acceptable carriers, excipients, binders, diluents, or the like. The oral drugs are produced in a form that can be ingested orally. The oral drugs include, for example, foods, granules, powders, tablets (including sugar-coated tablets), pills, capsules, syrups, emulsions, suspensions, and the like.

The oral drugs can be prepared with pharmaceutically acceptable additives. The pharmaceutically acceptable additives include, for example, excipients, carriers, disintegrators, binders, lubricants, buffers, coating agents, thickeners, tinctions, stabilizers, emulsifiers, dispersants, suspending agents, preservatives, perfumes, and the like. The excipients include lactose, sucrose, starch, mannitol, and the like, for example. The carrier include, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The disintegrators include calcium carbonate, calcium carboxymethylcellulose, and the like, for example. The binders include, for example, pregelatinized starch, acacia, carboxymethyl cellulose, polyvinylpyrrolidone, hydroxypropyl cellulose, and the like. The lubricants include, for example, talc, magnesium stearate, polyethylene glycol 6000, and the like. The buffers include phosphate, citrate, and the like, for example. The coating agents are added, for example, for the purpose of masking the taste or for the purpose of ensuring enteric solubility or persistence. The coding agents include, for example, ethyl cellulose, hydroxymethyl cellulose, polyoxyethylene glycol, cellulose acetate phthalate, hydroxypropylmethyl cellulose phthalate, eudragit (methacrylic acid, acrylic acid copolymer), and the like.

The oral drugs are prepared by compression molding the powdered PHB added with the excipients, disintegrators, binders or lubricants (talc, magnesium stearate, polyethylene glycol 6000, and the like), first. Subsequently, the compression-molded powdered PHB is coated with the coating agent, if necessary.

### [Preparation of Oral Drugs]

The oral drugs containing the powdered PHB can be included in health foods for humans and animals or pet therapeutic diets. Various proteins, sugars, fats, trace elements, vitamins, and the like may be mixed together with the powdered PHB into the health foods and the like. The health foods and the like may be liquid, semi-liquid, or solid, or may be a paste. The health foods and the like can be in the form of regular foods or nutritional supplements such as dietary supplements.

The packaging of the health foods and the like may display their function for treating, preventing, or ameliorating a disease or condition that can be treated, prevented, or ameliorated by increasing the ketone body concentration, or may display their antioxidant, detoxification, or anti-inflammatory capabilities. The health foods and the like may be beverages, and sugars, flavors, fruit juices, food additives, and the like used in the production of ordinary beverages may be added as appropriate. Foods according to the present disclosure may take a variety of forms, and may be produced according to known pharmaceutical production techniques. In this case, the above-mentioned additives can be used.

When the oral drugs or foods containing the powdered PHB are administered or ingested, the dosage or intake of the powdered PHB can be determined depending on the age, weight, symptoms, administration time, dosage form, administration method, combination of agents, or the like of the subject to be treated. For example, when the powdered PHB according to the present disclosure is administered as a health food, the effective amount of the powdered PHB can be administered in the range of 10 to 2000 mg/kg of body weight (preferably 100 to 1000 mg/kg of body weight) per adult human, once a day or divided into several dosage units. It should be noted that these dosages or intakes can be expressed by assuming that the body weight of an adult is 60 kg, and calculating the dosages or intakes of PHB per day for one adult weighing 60 kg as necessary.

### [Method for Synthesizing Powdered PHB]

PHB is a polymer of (R)-3-hydroxybutyric acid (ketone bodies) through ester bonding. PHB can be synthesized by fermentation or chemical synthesis. When the chemical synthesis method is used, the cost for synthesis will be high because expensive (R)-3-hydroxybutyric acid is used as a raw material. On the other hand, in the fermentation method using microorganisms, an inexpensive raw material including sugar is efficiently used for biosynthesis, and a large amount of PHB can be easily prepared.

### [Synthesis of PHB by Fermentation]

PHB is synthesized by fermentation using bacteria. Microorganisms capable of synthesizing PHBs include Halomonas, Bacillus, Azotobactor, Rhizobium, Vibrio, Chromobacterium, Pseudomonas, Micrococcus, Sphaerotailus, Hydrogenomonas, Cupriavidus, Rhodospirillum, Rhodopseudomonas, Chromatium, Spirillum, Comamonas, Aspergillus, Variovorax, Alcaligenes, and Ralstonia.

A composition of a culture solution for producing the powdered PHB may be prepared by combining one or more organic carbon sources and one or more nitrogen sources with minerals suitable for each microorganism. Examples of the organic carbon sources include glucose, fructose, mannose, galactose, xylose, arabinose, sucrose, maltose, cellobiose, citric acid, lactic acid, butyric acid, gluconic acid, ethanol, glycerol, and the like. Examples of the nitrogen sources include nitrates (sodium, potassium, calcium, etc.), nitrites, ammonium chloride, ammonium nitrate, ammonium carbonate, ammonium sulfate, urea, etc. The powdered PHB can be, for example, an alcohol suspension (e.g., a methanol suspension, an ethanol suspension), or an ether suspension as a pharmaceutically acceptable solvate or suspension.

The composition of the culture solution is, for example, 12.6 g of sodium hydrogen carbonate, 5.3 g of sodium carbonate, 2.0 g of potassium hydrogen phosphate, 1.0 g of salt, 12.5 g of sodium nitrate, 1.0 g of potassium sulfate, 40 mg of magnesium sulfate heptahydrate, 10 mg of calcium chloride dihydrate, 10 mg of ferric sulfate heptahydrate, and 80 mg of disodium edetate with respect to one liter of distilled water. The culture solution may contain 5% w/v of glucose. The culture solution may be added during the culturing of bacteria, as needed. Halomonas may be added, and the aerobic culture may be grown for 3 to 4 days, while being kept at 30° C.

Bacterial cells are cultured in prepared culture solution. During the culturing, the bacterial cells accumulate PHB within the cells. The culture solution after an aerobic culture contains PHB granules, water, and inorganic ions (nitrates, sodium, and the like) accumulated within the bacterial cell. Halomonas sp. OITC1261 produces ketone bodies at the same time as PHB, but ketone bodies are released outside the bacterial cell (in the culture solution) and are therefore removed later on in a PHB purification process. In OITC 1261, the accumulation of PHB granules reaches up to about 70% of a cytoplasm.

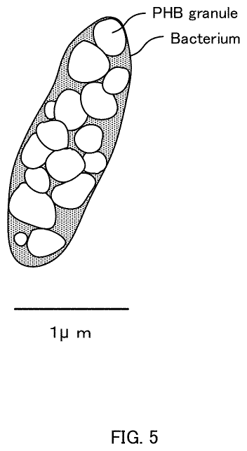

FIG. 5 schematically shows a bacterium accumulating PHB granules. Since PHB has a very long chain structure, it is highly folded in the bacterial cell and exists as a granular structure of tens to hundreds of nanometers.

### [Types of Purification of Powdered PHB]

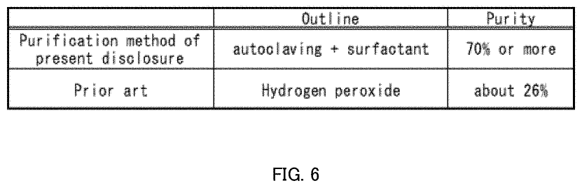

FIG. 6 shows an outline of a plurality of methods for purifying PHB. When the Soxhlet extraction method using organic solvents such as chloroform is employed, there is a risk of organic solvent residue. Therefore, the purification method of the present disclosure is a combination of autoclaving and treatment using a surfactant. By using the purification method of the present disclosure, the purity of the powdered PHB can be 70% or more. On the other hand, the prior art uses hydrogen peroxide, and the purity of the purified powdered PHB is only about 26%.

### [Method for Purifying Powdered PHB]

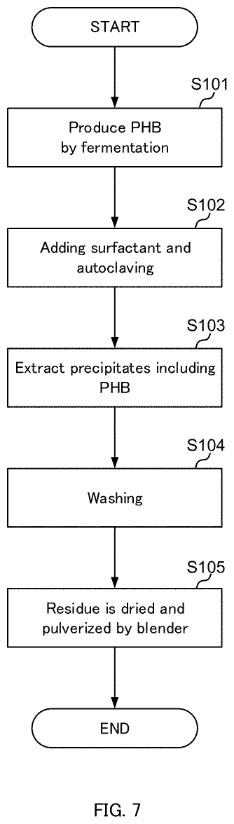

FIG. 7 is a flowchart showing an outline of the method for purifying the powdered PHB of the present disclosure. First, PHB granules are produced within Halomonas bacteria by fermentation using bacteria (step S101). Subsequently, a surfactant is added to the culture solution containing the bacteria, and then the culture solution is autoclaved several times (step S102). At this time, the powdered PHB having the weight-average molecular weight of about 700,000 can be obtained by using PHB derived from the genus Halomonas, adding less than 1% of the surfactant, and subjecting the resulting mixture to several autoclaves. Further, the powdered PHB having the weight-average molecular weight of about 590,000 can be obtained by using PHB derived from the genus Halomonas, adding 1% to 2% of the surfactant, and subjecting the resulting mixture to several autoclaves. In this step, bacterial components other than PHB are solubilized in an aqueous solution, and PHB precipitates as an insoluble component.

Next, the PHB granules are precipitated by centrifuging the insoluble components including PHB at 1000 rpm for 10 minutes, and the supernatant liquid is removed to extract the precipitated insoluble components including the PHB granules (step S103). Alternatively, instead of extracting the precipitated PHB granules by centrifugation, a method using a Vacuum Leonider in which the solution is heated and the volume of the solution is reduced by depressurizing a container containing the solution after heating may be used to concentrate the bacteria. The insoluble components containing PHB granules may be extracted using the filter press method in which the solution pressurized by a pressurizer is filtered after performing the method using the Vacuum Leonider.

The PHB granules taken out as precipitates are washed with water (step S104). Instead of water, ethanol or a mixture of water and ethanol can be used for washing. This washing allows the bacterial components other than PHB to be removed by dissolving them in water. By repeating step S104, the purity of the powdered PHB can be 90% or more.

The collected residue is dried, and the dried residue is crushed in a blender to synthesize the powdered PHB (step S105). By subjecting the bacteria to autoclaving or heat-drying treatment, cell membranes of the bacteria are destroyed and PHB granules in the bacteria (with the average polymerization degree of more than 10,000), which had formed a higher-order structure by weak intermolecular force or hydrogen bonding, can be turned into the powdered PHB with an average polymerization degree of about several thousand. PHB linear chains of the PHB granules in the bacteria associate by weak intermolecular forces and hydrogen bonding to form a higher-order structure, whereas the PHB linear chains of the powdered PHB exist without association.

### [Activation of Intestinal Bacteria]

The oral drugs containing the powdered PHB are administered for the purpose of treating constipation. It has been reported that when pigs were fed PHB (weight-average molecular weight: 840,000) and raised for five days, their defecation frequencies increased and bowel movements were improved (PCT International Publication No. 2005/021013). It has been reported that when the pigs were fed PHB for four weeks, the amount of volatile fatty acids, hydrogen sulfide, and mercaptans in pig excrement were reduced, and the odor components from the excrement were significantly improved (PCT International Publication No. 2005/021013). These results indicate that PHB improved the colon flora. That is, PHB increases the butyric acid production in the large intestine by increasing the number of bacterial groups that are capable of producing the butyric acid and belonging to Clostridium cluster IV, Clostridium cluster XIVa, and Clostridium cluster XVIII, such as Eubacterium, Roseburia, Coprococcus, Faecalibacterium, Ruminococcus, Lachnospira, Clostridium, and the like. Thus, the first target of PHB is considered to be intestinal bacteria, and the oral drugs containing the powdered PHB are considered to have therapeutic effects on constipation. In particular, administration of PHB having a weight-average molecular weight of between 10,000 and 700,000 to pigs is expected to improve the bowel movements in the pigs in a relatively short time.

### EXAMPLES

### Example 1: Synthesizing PHB by Fermentation

In Examples 1 and 2, the powdered PHB was produced by the purification method described above (FIG. 7). PHB was fermented using Halomonas sp. OITC1261 to synthesize PHB having a polymerization degree of several thousands to several tens of thousands. The culture solution was composed of, for example, 12.6 g of sodium hydrogen carbonate, 5.3 g of sodium carbonate, 2.0 g of potassium hydrogen phosphate, 1.0 g of salt, 12.5 g of sodium nitrate, 1.0 g of potassium sulfate, 40 mg of magnesium sulfate heptahydrate, 10 mg of calcium chloride dihydrate, 10 mg of iron (II) sulfate heptahydrate, and 80 mg of disodium edetoate being mixed into one liter of distilled water.

### Example 2: Purifying Powdered PHB

The culture solution had 1% to 2% of the surfactant added thereto and was then subjected to several autoclave treatments (1.2 atm, 120° C., 20 min, humidity: 100%) to sterilize the culture solution containing bacteria with pressure. Next, the insoluble components containing PHB were precipitated by centrifugation at 10000 rpm for about 10 minutes, and the solution was discarded. The PHB granules taken out as the insoluble components were washed with water.

The collected residue was dried at 100° C. for two hours. The dried residue was crushed in a blender to prepare the powdered PHB, and the powdered PHB was autoclaved to produce the powdered PHB having an average polymerization degree of about several thousands.

### Example 3: An Increase in Ketone Body Concentration Due to Ketone Ester and PHB

PHB was extracted from OITC1261 by the method for purifying the powdered PHB shown in Example 2 (purity: 90%, weight-average molecular weight: 590,000). Ketone ester and PHB whose amounts were adjusted such that the intake thereof was 500 mg/kg of body weight were mixed uniformly in yogurt (200 g) and fed to humans. The ketone body concentration in the blood was measured using a ketone body value electrode (FS Precision ketone measurement electrode) of Precision Exceed (Abbott) every other hour. After ketone bodies were confirmed to be stable, plain yogurt was ingested.

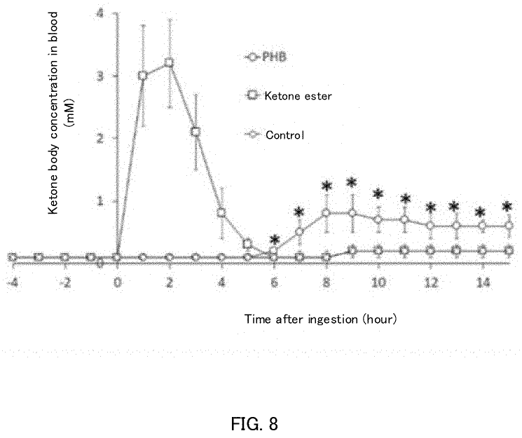

FIG. 8 shows measurement results of the ketone body concentration in the blood. The horizontal axis of the graph in FIG. 8 represents time. The vertical axis represents the ketone body concentration in the blood. Squares represent changes in the ketone body concentration in the blood over time in a group that ingested plain yogurt mixed with ketone ester. Circles represent changes in the ketone body concentration in the blood over time in a group that ingested plain yogurt mixed with the powdered PHB (purity of the powdered PHB: 90%). Diamonds represent changes in the ketone body concentration in the blood over time in a group that ingested only the plain yogurt.

It can be seen that in the group that ingested ketone ester, the ketone body concentration in the blood rapidly increases, but rapidly returns to the original level in about five hours after ingestion. On the other hand, in the group that ingested PHB, it was found that the ketone body concentration increased slowly from about five hours after ingestion and level of the concentration was sustained to at least 15 hours after the ingestion. Stars (*) on the graph after the sixth hour indicate the ratio of risk that the hypothesis being wrong is less than 5% when the experiment is repeated under the same condition, the hypothesis is that the group that ingested PHB has higher ketone body concentrations than the ketone ester group, that is, the group that ingested PHB has significantly higher ketone body concentrations than the group that ingested ketone ester. After the sixth hour, the ketone body concentration of the group that ingested PHB is significantly higher than that of the group that ingested ketone ester, indicating that PHB increases the ketone body concentration in the blood for a longer period of time than ketone ester.

### Example 4: Increase in Ketone Body Concentration Depending on Purity of PHB

The powdered PHB and ketone bodies whose amounts were adjusted such that the intake thereof was 500 mg, 300 mg, 100 mg, or 0 mg/kg of body weight were mixed uniformly in yogurt (200 g) and fed to humans. Said powdered PHB had been derived from OITC1261 and extracted by the purification method shown in Example 2 (purity: 70%, weight-average molecular weight: 590,000). The ketone body concentration in the blood over time was measured using a ketone body value electrode (FS Precision ketone measurement electrode) of Precision Exceed (Abbott) every other hour. After five hours from ingestion, ketone bodies were confirmed to be stable and then the plain yogurt was ingested.

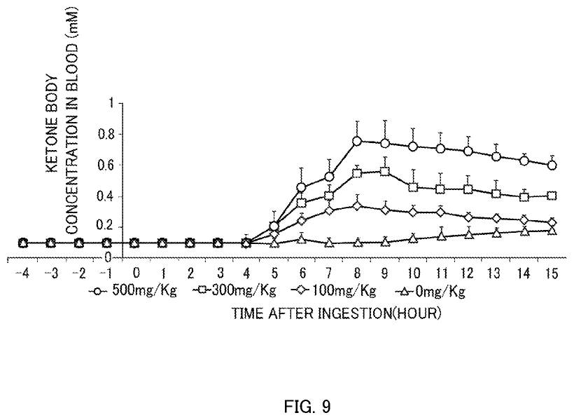

FIG. 9 shows measurement results of the ketone body concentration in the blood after ingesting the plain yogurt containing the powdered PHB. The horizontal axis of the graph of FIG. 9 represents time. The vertical axis represents the ketone body concentration in the blood. In the case where the ingestion amount of ketone bodies was 0 mg/kg of body weight (triangles shown in FIG. 9), humans were fed only the plain yogurt (200 g). It was found that, in the group that ingested PHB, the ketone body concentration increased slowly from about five hours after ingestion and level of the concentration was sustained to at least 15 hours after the ingestion. After six hours from ingestion, the group that ingested PHB had significantly higher ketone body concentrations in the blood than that of the control group, with the ratio of risk that the hypothesis is wrong being less than 5%. Therefore, it can be seen that PHB continuously increases the ketone body concentration in the blood. The larger the intake of ketone bodies per kilogram of body weight, the larger the increase in the ketone body concentration in the blood.

### Example 5: Increase in Ketone Body Concentration by Daily Intake of PHB

The plain yogurt (200 g) mixed with the powdered PHB (purity: 70%, weight-average molecular weight: 590,000) derived from OITC1261 and extracted by the purification method shown in Example 2 was fed to humans every morning at 9:00 am. PHB whose amount was adjusted such that the intake of PHB was 500 mg/kg of body weight was mixed uniformly in yogurt (200 g). Breakfast, lunch, and dinner were taken as usual. The ketone body concentration in the blood was measured using a ketone body value electrode (FS Precision ketone measurement electrode) of Precision Exceed (Abbott) every other day.

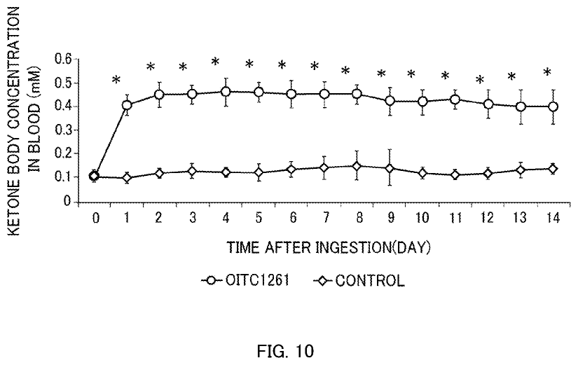

FIG. 10 shows measurement results of the ketone body concentration in the blood when the yogurt containing the powdered PHB was ingested daily. The horizontal axis of the graph of FIG. 10 represents the number of days. The vertical axis represents the ketone body concentration in the blood. Circles represent the group that ingested plain yogurt mixed with powdered PHB. Diamonds represent the control groups that ingested only the yogurt. From day 1 onward, the ketone body concentration in the blood sustained at 0.4 mM to 0.5 mM. The results showed a significant difference between the control group and the groups that ingested PHB with the ratio of risk being less than 5%.

This indicates that PHB has the ability to increase the ketone body concentration in the blood after day 1. On the other hand, according to PCT International Publication No. 2008/120778, when the powdered PHB (purity: about 26%, weight-average molecular weight: about 840,000) produced by the method described in PCT International Publication No. 2008/120778 is used, the increase in the ketone body concentration in the blood cannot be detected until day 14. These results suggest that the time required for the ketone body concentration in the blood to begin to increase varies greatly depending on the weight-average molecular weight and/or the purity of the powdered PHB. From these results, it can be inferred that the weight-average molecular weight of the powdered PHB should be less than 700,000, and preferably less than 590,000. Further, from these results, it can be inferred that the purity of the powdered PHB should be more than 50%, and preferably more than 70%.

### Example 6: Adjuvant Effect by Ketone Donors (COS7 Cell)

COS7 cells, that are a renal cytoblastoma, were used. Subconfluent cells were spread on 24-well plates at a density of 40,000 cells/cm² and cultured in Dulbecco's Modified Eagle Medium (D-MEM) containing 10% of inactivated fetal bovine serum for one hour. Various concentrations of ketone bodies (HB) and ketone ester (KE) were added to the culture medium of COS7 cells and cultured for 24 hours. After 24 hours of culturing, the culture medium was removed, and COS7 cells were cultured for two hours in PBS with 1 mg/ml of a chromogenic reagent 3-(4,5-dimethylthiazol-2-yl)-2,5-diphenyl tetrazolium bromide (MTT, Wako Pure Chemical Corporation).

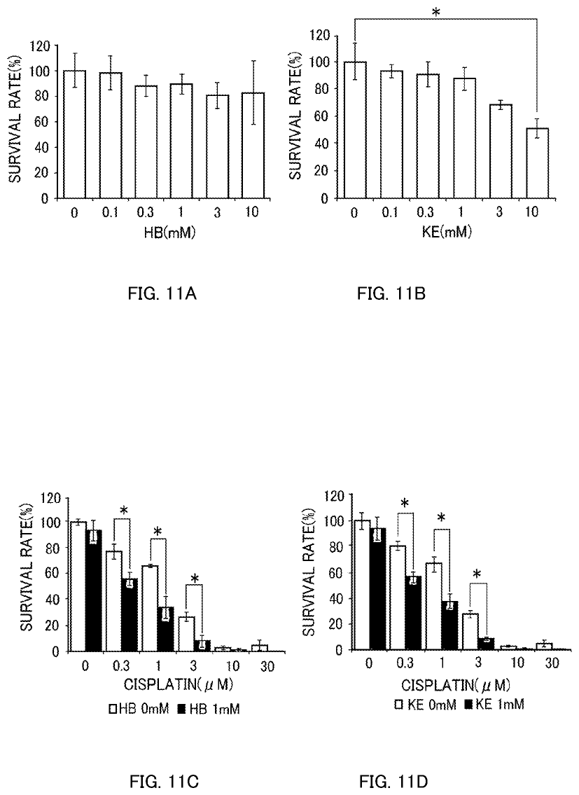

After the culturing, cell lysis solution (50% formamide, 20% sodium dodecyl sulfate) was added and left for 24 hours. Thereafter, the absorbance at 540 nm was measured using an absorbance measurement device (Biorad microplate reader), and the measured absorbance was used to calculate survival rates of COS7 cells. FIGS. 11A to 11D show the survival rates of COS7 cells when ketone bodies (HB) or ketone ester (KE) was added. In the examples in FIGS. 11A and 11B, the toxic effects of ketone bodies (HB) and ketone ester (KE) on COS7 cells were examined, respectively. Since the powdered PHB is hydrolyzed into ketone bodies by intestinal bacteria in the large intestine, the toxic effect of ketone bodies on COS7 was verified.

FIG. 11A shows the survival rates of COS7 cells when ketone bodies were added, and FIG. 11B shows the survival rates of COS7 cells when ketone ester was added. The horizontal axis of FIGS. 11A and 11B represents the concentrations of the added ketone bodies and ketone ester, respectively. The vertical axis of FIGS. 11A and 11B represents the survival rates of COS7 cells determined from the measured absorbance. Significant difference tests were carried out with Student t-TEST at a risk ratio of 5%, and significant differences were marked with stars (*). Neither of the ketone donors was toxic at least up to 1 mM of the concentration.

Various concentrations of ketone bodies (HB) and ketone ester (KE) was added to the medium of COS7 cells and the COS7 cells were cultured for 1 hour, and various concentrations of cisplatin were added thereto. Thereafter, the process was performed in the same manner as in the examples of FIGS. 11A and 11B. The adjuvant effect of 1 mM of ketone bodies and ketone ester on toxicity caused by the anticancer agent cisplatin was verified.

FIGS. 11C and 11D show the survival rates of the COS7 cells when ketone bodies (HB) or ketone ester (KE) was added together with cisplatin. FIG. 11C shows the survival rates of COS7 cells when 0 mM (white) or 1 mM (black) of ketone bodies were added. FIG. 11D shows the survival rates of COS7 cells when 0 mM (white) or 1 mM (black) of ketone ester was added. The horizontal axis of FIGS. 11C and 11D represents the concentration of cisplatin added.

Cisplatin is toxic to COS7 cells at a dosage between 0.3 and 10 μM, and the median lethal dose is about 2 μM. Significant difference tests were carried out with Student t-TEST at a risk ratio of 5%, and significant differences were marked with stars (*). The significant difference tests were carried out between a ketone body and ketone ester treated group (black) and an untreated group (white). 1 mM of ketone bodies and ketone ester significantly promoted the toxic effect caused by cisplatin. That is, ketone bodies and ketone ester had the adjuvant effect. Considering that PHB is hydrolyzed by intestinal bacteria to produce ketone bodies, PHB may act as an adjuvant to cisplatin to inhibit cancer growth.

### Example 7: Adjuvant Action by Ketone Donor (Hela Cell)

Instead of COS7 cells used in Example 6, Hela cells, a uterine myoblastoma, were used as cancer cells, and the same test as in Example 6 was conducted. Significant difference tests were carried out with Student t-TEST at a risk ratio of 5%, and significant differences were marked with stars (*). Toxic effects of ketone bodies (HB) and ketone ester (KE) on Hela cells were examined.

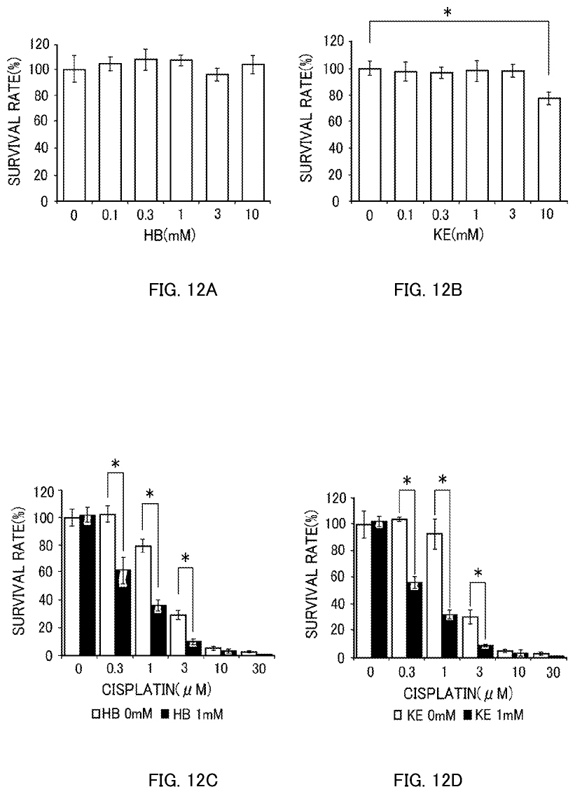

FIGS. 12A to 12D show survival rates of Hela cells when ketone bodies (HB) or ketone ester (KE) was added. FIG. 12A shows the survival rates of Hela cells when ketone bodies were added, and FIG. 12B shows the survival rates of Hela cells when ketone ester was added. The horizontal axis of FIGS. 12A and 12B represents the concentrations of the added ketone bodies and ketone ester, respectively. The vertical axis of FIGS. 12A and 12B represents the survival rates of Hela cells. As shown in FIGS. 12A and 12B, neither the ketone bodies nor the ketone ester was toxic at least up to 1 mM of the concentration.

The adjuvant action when 1 mM of ketone bodies or ketone ester was added together with the anticancer agent cisplatin was examined. FIGS. 12C and 12D show the survival rates of Hela cells when 1 mM of ketone bodies or ketone ester was added together with anticancer agent cisplatin. FIG. 12C shows the survival rates of Hela cells when 0 mM (white) or 1 mM (black) of ketone bodies were added. FIG. 12D shows the survival rates of Hela cells when 0 mM (white) or 1 mM (black) of ketone ester was added. The horizontal axis of FIGS. 12C and 12D represents the concentration of the added cisplatin.

Cisplatin is toxic to Hela cells at the dosage between 0.3 and 10 μM, and the median lethal dose is about 2 μM. The significant difference tests were carried out between the ketone body or ketone ester treated group (black) and the untreated group (white). Significant difference tests were carried out with Student t-TEST at a risk ratio of 5%, and significant differences were marked with stars (*). As represented by the stars (*), when 0.3-3 μM of cisplatin was added, both the 1 mM ketone body treated group and the 1 mM ketone ester treated group significantly promoted the toxic effects due to cisplatin as compared to the untreated group. That is, ketone bodies and ketone ester had adjuvant effects.

### Example 8: Increase in Ketone Body Concentration in the Blood by PHB

The changes in ketone body concentration in the blood of mice fed PHB powder were examined. First, the mice were transferred to a test environment and acclimated for one week or more. Mice were fed diets containing 0.2% of PHB (the powdered PHB equivalent to 0.2% of the weight of the diet) or 2% of PHB (PHB equivalent to 2% of the weight of the diet). At each time point, tails of the mice were cut off and the ketone body concentration in the blood was measured. Here, the powdered PHB has a purity of 95%, and the weight-average molecular weight of PHB is 590,000.

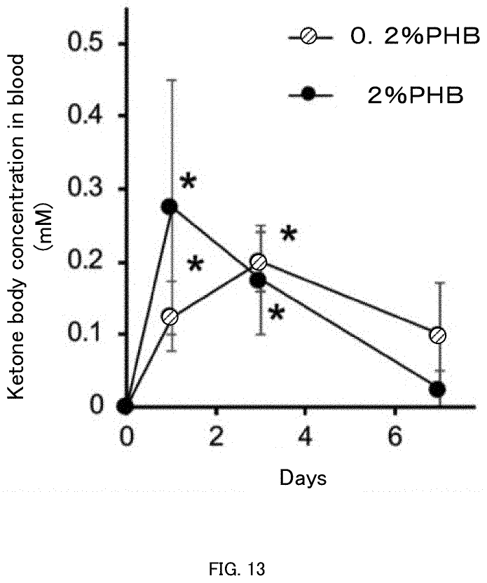

FIG. 13 shows measurement results of the ketone body concentration in the blood of the mice fed with the powdered PHB. The horizontal axis of FIG. 13 represents the time after being fed the diet containing PHB. The vertical axis represents the ketone body concentration in the blood. Black circles represent changes in ketone body concentration in the blood when being fed the diets with 2.0% of PHB. Hatching circles represent changes in ketone body concentration in the blood when being fed the diets with 0.2% of PHB. The purity is the ratio of the mass of PHB to the mass of the diet.

As shown in FIG. 13, when being fed the diets with 0.2% of PHB, the ketone body concentration in the blood reached the maximum in three days and then decreased. The ketone body concentration in the blood when being fed the diets with 2.0% of PHB reached the maximum in one day and then decreased. The maximum ketone body concentration was higher when PHB having the purity of 2.0% was fed than when PHB having the purity of 0.2% was fed, but it was confirmed that the ketone body concentration in the blood increased in both cases when PHB with purities of 2.0% and 0.2% was fed. As described above, even if the purity of PHB is relatively low, the effect of increasing the ketone body concentration in the blood is obtained. When the mice were fed the diets continuously every day, it is assumed that a constant concentration is sustained as shown in FIG. 10. In addition, it is assumed that the ketone body concentration in the blood of the mice fed the diets containing PHB having the purity less than 0.2% cannot be distinguished from the ketone body concentration in the blood of the mice fed the diets without PHB.

### Example 9: Confirmation of Inhibitory Effect of PHB on Cancer Growth

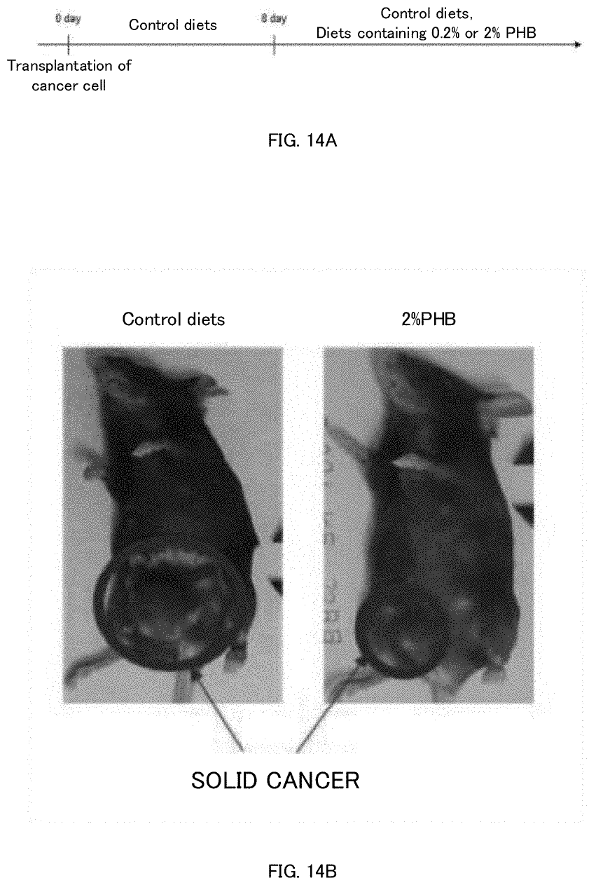

Mouse breast cancer cells, that are E0771 cells, were used. The cells were cultured in Dulbecco's Modified Eagle Medium (D-MEM) containing 10% of inactivated fetal bovine serum, and after sufficient growth, 2 million E0771 cells per mouse were transplanted into mammary gland tissue of C57BL6 mice. FIGS. 14A and 14B show a confirmation test of inhibitory effects of PHB on cancer growth. FIG. 14A shows a protocol for feeding the mice. FIG. 14B shows pictures of solid cancer around breasts of mice after transplanting cancer. As shown in FIG. 14A, after transplantation of the breast cancer cells, the control diets without PHB were fed to mice for eight days. The mice were divided into two groups: one group was fed the control diets continuously after day 9, and the other group was fed the diets containing 0.2% of PHB or 2.0% of PHB from day 9. There was no significant difference in weight change between these groups.

The picture on the left side of FIG. 14B shows the cancer in a mouse fed the control diet continuously after day 9 after the transplantation of cancer cells. The picture on the right side of FIG. 14B shows the cancer in a mouse fed the diet containing 2.0% of PHB (2% PHB) from day 9 after the transplantation of cancer cells. As shown by circles around the breasts of the mice in FIG. 14B, the size of solid cancer in the mouse in the PHB group is clearly smaller than that in the mouse in the control diet group.

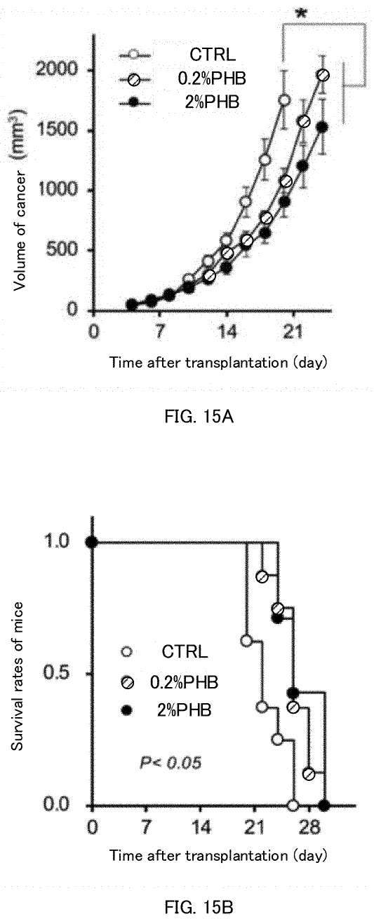

FIGS. 15A and 15B are graphs showing the inhibitory effect of PHB on cancer growth. FIG. 15A shows the transition of the volume of the cancer transplanted into the mice. FIG. 15B shows the survival rates of the mice after the transplantation of breast cancer cells. In FIG. 15A, the horizontal axis represents the time after the transplantation of cancer cells, and the vertical axis represents the volume of cancer. White circles (CTRL in FIG. 15) represent the group that fed the control diet after day 9. Hatching circles represent the group that fed 0.2% of PHB after day 9. Black circles represent the group that fed 2.0% of PHB after day 9.

In the group that fed the control diet, the volume of cancer rapidly increased after day 10. On the other hand, in group that fed 2.0% of PHB, an increase in the volume of cancer was significantly inhibited after day 14. In the group that fed 0.2% of PHB, the increase in the volume of body cancer was significantly inhibited after day 16. These results confirmed that PHB has an effect of inhibiting cancer growth.

The horizontal axis of FIG. 15B represents the time after the transplantation of cancer cells, and the vertical axis represents the survival rates of the mice. The mice in the group that were fed the control diet (CTRL) began to die after day 20, and all died by about day 26. The median survival period of the mice in the control diet group was 22 days. The median survival period is the period of time when the survival rate is 50%. On the other hand, the median survival period of the mice in the 2% PHB and 0.2% PHB groups was 26 days, which was 4 days longer than that of the mice in the control diet group. These results confirmed that feeding PHB to mice significantly inhibited cancer growth.

As shown in FIGS. 15A and 15B, cancer growth inhibition was observed even when the purity of PHB was relatively low, 0.2%. This suggests that there is a pathway that leads to cancer growth inhibition without going through an increase in the ketone body concentration in the blood, and PHB may be considered to also lead to cancer growth inhibition through activation of the receptor HCAR2. On the other hand, as shown in FIG. 4, it is assumed that the powdered PHB produces the butyric acid with the intestinal bacteria, the butyric acid stimulates macrophages, and activation of the macrophages stimulates Treg cells (regulatory T cells) to suppress hyperimmune to cancer cells. That is, an oral administration of the oral drug containing the powdered PHB inhibits cancer. In addition, it is inferred to have a cancer-inhibiting effect when the oral drug is used with the radiotherapy or anti-cancer agents.

The present invention has been described above on the basis of the exemplary embodiments. The technical scope of the present invention is not limited to the scope explained in the above embodiments, and it is obvious to those skilled in the art that various changes and modifications within the scope of the invention may be made. An aspect to which such changes and modifications are added can be included in the technical scope of the present invention is obvious from the description of the claims.

The oral drug, the adjuvant cancer therapy, and the pet therapeutic diet of the present embodiment can bring about therapeutic effects on diseases such as cancer because they sustain the increased ketone body concentration in the blood for a longer period of time.

What is claimed is:

1. An oral drug comprising:
powdered poly (R)-3-β-hydroxybutyric acid having a weight-average molecular weight of 10,000 to 700,000.

2. The oral drug according to claim 1, comprising:
the powdered poly (R)-3-β-hydroxybutyric acid having (a) a purity of 70% or more and (b) a weight-average molecular weight of 10,000 to 590,000.

3. The oral drug according to claim 1, comprising:
the powdered poly (R)-3-β-hydroxybutyric acid having (a) a purity of 90% or more and (b) a weight-average molecular weight of 10,000 to 590,000.

4. An oral drug for increasing a ketone body concentration in blood comprising:
powdered poly (R)-3-β-hydroxybutyric acid having a weight-average molecular weight of 10,000 to 700,000.

5. An oral drug for activating intestinal bacteria in the large intestine comprising:
powdered poly (R)-3-β-hydroxybutyric acid having a weight-average molecular weight of 10,000 to 700,000.

6. An oral drug for activating macrophages in the large intestine comprising:
powdered poly (R)-3-β-hydroxybutyric acid having a weight-average molecular weight of 10,000 to 700,000.

7. An adjuvant cancer therapy comprising:
powdered poly (R)-3-β-hydroxybutyric acid having a weight-average molecular weight of 10,000 to 700,000.

8. The adjuvant cancer therapy according to claim 7, comprising:
the powdered poly (R)-3-β-hydroxybutyric acid has a purity of 50% or more.

9. The adjuvant cancer therapy according to claim 7, comprising:
the powdered poly (R)-3-β-hydroxybutyric acid has a purity of 90% or more.

10. A pet therapeutic diet to be provided to subjects during or after treatment of cancer comprising:
powdered poly (R)-3-β-hydroxybutyric acid having a weight-average molecular weight of 10,000 to 700,000.
